(19) United States
(12) Patent Application Publication
(10) Pub. No.: US 2004/0010237 A1
(43) Pub. Date: Jan. 15, 2004

(54) Novel needle for medical device and device comprising same

(76) Inventor: Bernard D'Ussel, Paris (FR)

Correspondence Address: JACOBSON HOLMAN PLLC, 400 SEVENTH STREET N.W., SUITE 600, WASHINGTON, DC, 20004, US

(21) Appl. No.: 10/296,227

(22) PCT Filed: May 31, 2001
(86) PCT No.: PCT/FR01/01695

(30) Foreign Application Priority Data
Jun. 5, 2000 (FR) 00 07137

(51) Int. Cl. A61M 5/32

(52) U.S. Cl. 604/272; 604/265

(57) ABSTRACT

The invention concerns a needle for medical use comprising a rounded end and provided with a tapered hub made of an inert material soluble in corporeal fluids, such as a sugar, a salt or any other like product. The invention is applicable in the medical field.

The invention relates to a novel needle for medical use and a device comprising it.

For several years, mainly as a result of contagious diseases such as aids and hepatitis C, hospital and other medical staff have been greatly exposed to contagion when handling used needles.

The present advice is to withdraw the needle after use by means of a box for holding it in order to protect the staff from pricks when handling it. However, these devices cannot ensure total safety, in particular against any accidental contact with staff between the moment when the needle is withdrawn from the patient's body and the moment when removed from the device as previously described or when the device and needle are thrown into a special box provided for such purpose.

It will be remembered that a syringe needle or any other device for injecting a liquid substance into a human or animal body should have a certain number of qualities. More particularly it should have a very sharp free end so that it can be inserted quickly, accurately and as painlessly as possible.

Therefore, one object of the present invention is to provide a needle for medical use with a sharp end which does not, after use, present any risk of pricking the medical staff.

Another object of the invention is to provide a said needle at a competitive minimum price.

An additional object of the invention is to provide a medical device equipped with a said needle.

These objects, together with others which will appear hereinafter, are achieved with a needle for medical use characterised according to the invention in that one end of the needle is rounded and is provided with a sharp tip.

Advantageously the sharp end is made of an inert material soluble in body fluids, such as a sugar, salt or other similar product.

The present invention also relates to a medical device such as a syringe or transfusion apparatus comprising a needle as defined hereinbefore.

The following description, which is in no way limitative, will give the s man skill in the art a clearer idea of how to work the invention and of the advantages thereof.

A needle for medical use according to the present invention comprises a body, generally of metal such as aluminum, and is formed with a duct along its axis for conveying the liquid for injection. Its free end, or distal end since furthest from the device on which the needle is mounted, is provided according to the invention with a sharp tip.

This sharp tip is made of an inert material, i.e. a material which does not react either with the liquids for injection or with the constituents of the human and/or animal body, and which dissolves very quickly in body fluids.

A said tip, for example, can be made of a sugar, salt or any similar product resistant to the sterilisation temperatures. The needle, like any medical equipment, must be sterilised before use.

A needle according to the invention is constructed by known production methods but without the sharpening step. As a result the distal end of the needle is rounded and does not constitute any risk of the staff.

A drop of the inert material is placed on the rounded end and acquires a sharp shape, forming the sharp tip.

In an embodiment, the rounded-end needle is vertically immersed, i.e. with the rounded end downwards, in a hot solution of the inert material.

When raised, the drop of inert material retained takes the shape of a sharp point during solidification.

During the actual puncture, whether subcutaneous, intramuscular or intravenous, the sharp tip serves exactly the same purpose as a sharp end on a needle but dissolves very quickly in body fluids, so that the duct in the needle is open at the said end and a substance can be injected.

1. A needle for medical use and comprising a sharp end, characterised in that one end of the needle is rounded and provided with a sharp tip.

2. A needle for medical use according to claim 1, characterised in that the sharp tip is made of an inert material soluble in body fluids, such as a sugar, salt or similar product.

3. A medical device characterised in that it comprises a needle according to claim 1 or 2.
